(19) United States
(12) Patent Application Publication
(10) Pub. No.: US 2008/0053202 A1
(43) Pub. Date: Mar. 6, 2008

(54) Devices, methods and systems for fuel monitoring

(76) Inventors: Vladimir Rohklin, Hamden, CT (US); Richard A. Deverse, Kailua-Kona, HI (US); Van Malan, Kailua Kona, HI (US); Paul Troy, Captain Cook, HI (US); Andreas C. Coppi, Groton, CT (US); Ronald R. Coifman, North Haven, CT (US); Frank Geshwind, Madison, CT (US); Ed Kearns, North Haven, CT (US); William G. Fateley, Manhattan, KS (US)

Correspondence Address: FULBRIGHT & JAWORSKI, LLP, 666 FIFTH AVE, NEW YORK, NY, 10103-3198, US

(21) Appl. No.: 11/786,830

(22) Filed: Apr. 13, 2007

Related U.S. Application Data
(60) Provisional application No. 60/791,764, filed on Apr. 13, 2006.

(51) Int. Cl. G01N 33/22; G01F 23/284; G01J 3/28

(52) U.S. Cl. 73/61.41; 356/326; 73/290 R

(57) ABSTRACT

Disclosed are devices, methods and systems for the measurement of fuel composition and the detection and measurement of phase separation in fuel storage tanks. An embodiment measures optical and/or electrical impedance spectral features of the fuel by a series of sensors placed at a series of heights within a fuel storage tank. Embodiments are disclosed to measure aspects of the water, ethanol and gasoline content as well as other constituents of a fuel mixture, as well as the onset of the two phase event and the completed extent of the water intrusion into the system in concert with the level at which the layers exist in a fuel container.

### RELATED APPLICATION

This application claims priority benefit under Title 35 U.S.C. §119(e) of provisional patent application No. 60/791,764 filed Apr. 13, 2006, which is incorporated by reference in its entirety.

### BACKGROUND AND FIELD OF THE INVENTION

The present invention relates generally to devices, methods and systems for measurement and monitoring of the chemical and physical properties of fuels in storage containers, fuel tanks, fuel lines and pipelines, and more specifically, in part, to the detection and measurement of “two phasing” in petrochemicals in storage containers and fuel tanks. Modern petrochemical fuels are often mixed with so called bio-fuels such as ethanol. These bio-fuels are sold in various percentages-by-volume mixes. These systems are inherently unstable and in the presence or upon the introduction of water will separate into a two-layer formation where the lower layer is comprised of the water and ethanol in the system and the upper layer is comprised primarily of the fuel such as gasoline, iso-octane fuel, etc.

This two phasing can cause a situation in which liquid pumped from the tank is either water and ethanol, which will not burn in standard gasoline engines, or perhaps more typically, the liquid pumped can be pure gasoline (if taken from the top), in which case one looses the benefit of the bio-additives to the fuels, including but not limited to compliance with environmental laws.

Two phasing is also temperature dependent. A fuel that is single phased at one temperature, may two phase at another temperature depending on the relative amounts of the constituents in the fuel mixture. This creates a particular problem when fuel is to be transferred from a first container to a second container. In particular, for gasoline ethanol mixtures, when the second container is at a lower temperature than the first container, two phasing may occur in the second container when it did not in the first. For example, the first container could be an underground storage tank at a gas station, and the second could be the gas tank of a car. Hence there is a need for devices, methods and systems that monitor fuel composition, water content and fuel temperature, for fuel in a first container, as well as the temperature in a second container, to alert a user when this condition of temperature related two phasing is imminent.

Sensing of the level of fluids and petrochemicals in containers is commonly used to keep track of inventory of fluid, monitor for leaks and in some cases test for water intrusion into the tank. In prior art systems, typically employing floats, gasoline and water layer separation can be measured, but the added complexity introduced by ethanol in the system causes such float based systems to fail, in part because the density of water-ethanol mixes is not the same as the density of water alone.

Additionally, even when two phasing has not occurred (due to surface tension and similar effects), a layer substantially comprised of water may sit at the bottom of a fuel tank. When this happens, the addition of more fuel can stir up the mixture, thus introducing the water into the fuel and causing two phasing. This is another reason why it is important to measure the level of fluids and petrochemicals in containers.

Consequently, there is a need for a system that measures aspects of the water, ethanol and gasoline content of a fuel mixture within a fuel system, as well as the onset of the two phase event and the completed extent of the water intrusion into the system in concert with the level at which the layers exist in a fuel container. Further, there is a need for devices, methods and systems that monitor fuel composition, water content and fuel temperature, for fuel in a first container, as well as the temperature in a second container, to alert a user when a condition of temperature related two phasing is imminent.

### OBJECT AND SUMMARY OF THE PRESENT INVENTION

It is an object of the present invention to measure the composition of fuels at a particular location within a fuel system such as a storage tank or gas line, and in particular, in some embodiments, to measure aspects of the water, ethanol and gasoline content of a fuel mixture within a fuel system, each in order to monitor the condition of the fuel system for phase separation events. In some embodiments of the present invention, as detailed herein, this is accomplished by measuring optical and/or dielectric properties of the fuel including but not limited to optical or electrical impedance measurements.

It is an object of the present invention to measure the composition of fuels as a function of the height within a fuel storage tank. In some embodiments of the present invention, as detailed herein, this is accomplished by measuring optical or dielectric spectral features of the fuel by a series of sensors placed at a series of heights within the storage tank. In some embodiments, individual sensors disposed vertically, that happen to sit at an interface between different phases/layers within the fuel storage tank, can be used to determine the level of the interface more precisely. This is accomplished, in part, by using the spectral properties of the fuel to determine the fraction F of phase 1 vs. phase 2 within the vertical sensor. Combined optionally with other information including but not limited to density data of the different phases, this information is used to determine the height of the interface as it is in direct proportion to the fraction F.

It is an object of the present invention to monitor fuel composition, water content and fuel temperature, for fuel in a first container, as well as the temperature in a second container, to alert a user when a condition of temperature related two phasing is imminent. In some embodiments of the present invention, as detailed herein, this is accomplished by measuring optical and/or dielectric properties of the fuel including but not limited to optical or electrical impedance measurements. In some embodiments, these measurements are further compared to empirical, tabulated or otherwise empirically or theoretically modeled phase diagrams for the system, in order to determine when the fuel in either container is likely to be near a phase region in which two phases can occur.

### BRIEF DESCRIPTION OF THE DRAWINGS

The present invention will be understood and appreciated more fully from the following detailed description, taken in conjunction with the drawings in which:

FIG. 1 shows a parallel plate capacitor with material between the plates, so that the capacitance of the capacitor will depend on the dielectric properties of the material

FIG. 2 shows a simplified schematic circuit for measuring capacitance.

FIG. 3 shows one embodiment of the present invention for measuring the composition of fuels as a function of the height within a fuel storage tank

FIG. 4 shows an embodiment of a device reader in accordance with an embodiment of the present invention

FIG. 5 shows one embodiment of the present invention for measuring the composition of fuels as a function of the height within a fuel storage tank

FIG. 6 shows prior art components for fuel monitoring.

FIG. 7 shows an embodiment in accordance with the present invention, comprised of modification of the components from FIG. 6.

### DETAILED DESCRIPTION OF THE EMBODIMENTS

Turning now to FIG. 1, when a material (100) such as but not limited to a fuel mixture, substantially occupies the space between two plates (110) and (120), then a circuit (130) disposed to measure the capacitance between the two plates will measure an amount that depends on the dielectric constant of the material (100). Note that material (100) is meant to illustrate the presence of a material or substance (100) between the plates (110) and (120). While the combination (100), (110) and (120) in the figure resembles in some ways the electronic schematic symbol for a crystal, this is not the intended meaning of these symbols in FIG. 1.

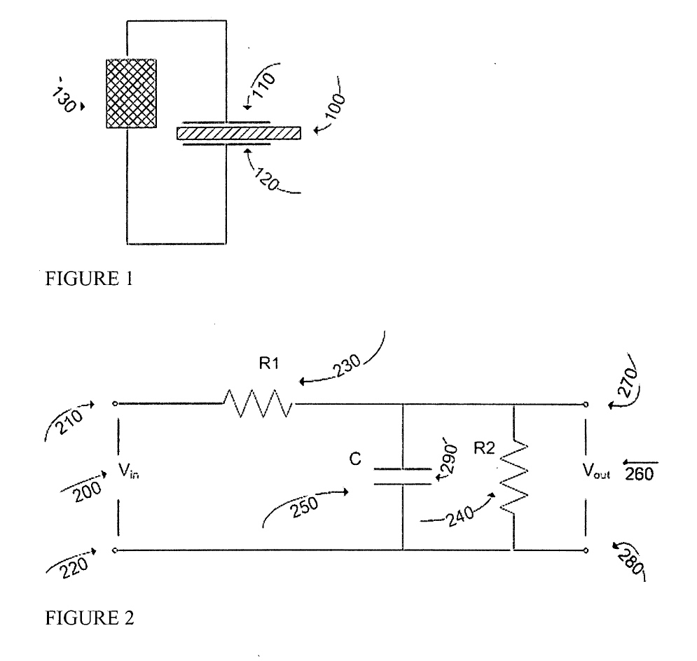

FIG. 2 shows a schematic of a capacitance measuring circuit. An input signal Vᵢₙ (200) is applied across input terminals (210) and (220). A circuit is comprised of resistors R1 (230), and R2 (240) and capacitor C (250), such capacitor including but not limited to a capacitive sensor as disclosed herein. An output signal Vₒᵤₜ (260) across output terminals (270) and (280) will have a voltage that, when compared with Vᵢₙ, will depend on the dielectric properties of the material (290) between the plates of the capacitor C (250). Indeed the impedance if the circuit across (270) and (280) will depend on both the capacitive and conductive impedances of the capacitor C (250), and importantly will also vary according to the frequency content f of the input signal Vᵢₙ (200). By varying the frequency content f through a predetermined range or otherwise as disclosed herein, an impedance spectrum of the material (290) is produced. The schematic presented in FIG. 2 is meant to be illustrative and not limiting. One of ordinary skill in the art will readily see that more sophisticated circuit designs can be used to estimate the capacitive and conductive impedance spectra of the material (290) as is standard in the art, and with choice of circuit suitable to particular application parameters such as desired precision and frequency range.

In some impedance spectroscopy measurement systems, the signal Vᵢₙ is taken to be a wave of pure frequency f. For each of a predetermined series of values for f, the impedance is measured and an impedance spectrum results. In one aspect of the present invention, more sophisticated waveforms are used to directly measure (impedance) spectral properties of interest of the material (290). Such waveforms include but are not limited to broadband pulses. In particular embodiments, a broadband pulse is applied at Vin, and then the waveform Vout is measured and digitized. A numerical Fourier transform (FFT) is then applied. The resulting waveform is compared with a reference waveform giving the analogous system response for the case when the space between the plates is, for example, filled with air (or in another example, in vacuum). Such comparison can include, but is not limited to, the pointwise numerical ratio computation, as a function of frequency. Other more sophisticated examples include, but are not limited to, direct measure waveforms. In particular, the methods of U.S. Pat. No. 6,859,275, “System and Method for Encoded Spatio-Spectral Information Processing”, by William G. Fateley et. al., are applicable, which is hereby incorporated herein by reference in its entirety.

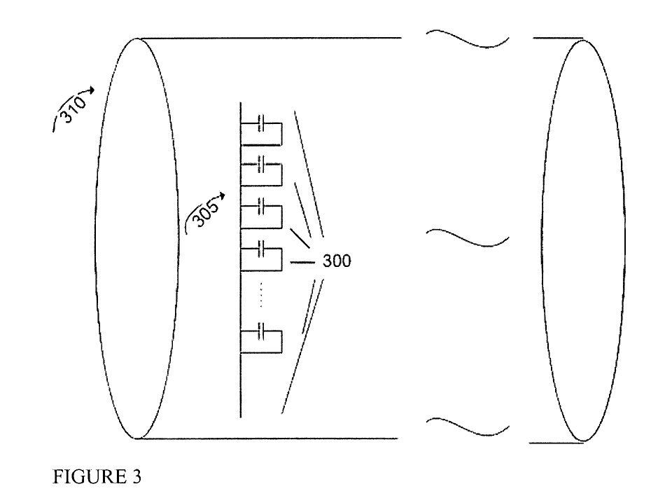

FIG. 3 shows an embodiment of the present invention for measuring the composition of fuels as a function of the height within a fuel storage tank. In the figure, a series of capacitive sensors (300) as disclosed herein, for example including but not limited to the type shown in FIG. 1, are placed at regularly spaced intervals along at least a portion of the height of a storage tank (310). Individual capacitive sensors within the set of sensors (300) (one example shown as 305), will have an impedance response according to the present invention, that provides information about the chemical composition of the fuel or air or other substance(s) between the plates of the sensor (305). In particular, the dielectric constant of air is about 1, gasoline about 2, alcohols such as ethanol about 20, and water about 80. Simply by measuring the capacitive reactance of the sensor (305) at one or two frequencies will allow for the discrimination between air, gasoline (and/or gasoline+additives such as ethanol), and ethanol+water mixtures, in accordance with the methods outlined herein but one method in particular consists of thresholding.

In an embodiment, a sensor comprises 16 metallic pads approximately 0.125″ apart from each other. The pads are situated in a linear fashion spaced consistently and substantially periodically along the length of the sensor active area. It will be appreciated by one of skill in the art that the spacing and/or size do not need to be contiguous or equal or of a certain geometric configuration in order to practice the present invention. However in some embodiments the uniform geometry described has the advantage of simplifying the analysis of the system. The capacitance between the various pairs of pads at different heights within the sensor, will correspond to the dielectric constant of the fluid between the pads. In this way, the device measures the level of various materials in a container.

In an embodiment, such a sensor employs two parallel circuit boards with large pads facing together. Each facing pair of pads is used to sense capacitance by means of a Capacitance to Digital Converter (CDC) like the one made by Analog Devices (AD7746). This particular device has two channels of input and is connected to four pads, two pairs of facing pads that are consecutively placed in the vertical orientation. A large array of capacitive sensing channels may be realized by arranging a number of CDC's linearly on the outside of the circuit boards. Each CDC communicates its results via I²C-bus. With the use of a bus multiplexer, like the Philips PCA9544A, many CDC's may be multiplexed to one I²C-bus channel. The current sensor has only four connections in the interface, power (+5), ground, data, and data clock (I²C-bus). The sensor is made with a multilayer board that has an internal ground plane and a power plane to reduce noise sensed at the capacitance pads induced by the serial communication, digital control, and any stray currents or capacitance in the environment. Two jumpers (RJ1 and RJ2) allow the two boards that make up a sensor to have unique I²C-bus addresses to allow the microcontroller to communicate.

In such an embodiment, the sensor is read or conditioned with a microprocessor, for example a Microchip PIC 18F452. In some embodiments, the microprocessor can be placed on a separate circuit board and connected to the sensor circuit. In some embodiments, this separate microprocessor board has power conditioning and a serial driver as well. The PIC has an I²C-bus peripheral that allows for connection to the sensor with cable lengths of many feet. The software in the processor continuously polls each CDC hi turn for the capacitance found at each pair of pads. The CDC has limited dynamic range for a single measurement (4 pF) and an internal Digital to Analog Converter (DAC) allows the sensor to change its range from 0 to 25 pF. In an embodiment, software is used to auto-range the DAC to adjust for current conditions. When a channel is being read, all other channels including their individual excitation sources are shut down as to not influence the reading of the channel of interest. Between each channel read, the PIC looks at the serial port for the presence of a command being received. If a command is present, the PIC sends all of the current channel results out the serial port in a stream. In an embodiment, the PIC is connected to a Bluetooth module that implements the serial port profile for communication with the HMI.

In some embodiments, it is of some use to coat the capacitive sensor system with a dielectric or insulating protective coating to protect the circuit parts from chemical or other interaction with the contents of the fuel tank. It will be appreciated by one of skill in the art that such coatings, used appropriately, will behave exactly like a virtual capacitor in series with the uncoated circuit, and thus it will be readily apparent how to modify the invention for use when such coatings are applied. Conversely, it will be understood that if one wants to avoid such coatings, for example to simplify manufacturing processes, the use of the system as disclosed herein, and the equivalent of these coatings from a system response point of view, can be handled by adding a series capacitance to the probe(s) within the system. For more sensitive measurements a precision capacitor in series with each capacitance probe is an alternative to insulating the probes since dielectric measurements will be affected by uneven deposition and surface chemistry. This alternative has advantages for some embodiments. By adding a capacitor in series, one can limit the capacitance to the range of values measurable by the circuit thus avoiding ‘out of range’ values caused by conductive fluids, or unexpected shorts.

By making this series capacitance a controllable and variable capacitor, certain advantages are obtained in some embodiments. In particular, because the dielectric constants of water and gasoline are so different, it will be helpful in some embodiments to be able to “auto-range” the capacitance of the probes, and this can be accomplished, for example, by use of the aforementioned variable series capacitor.

In one embodiment, a coating is used that is comprised of a flexible membrane made by POR-15 named ‘U.S. Standard Fuel Tank Sealer’. One method of application is to dip the completed and tested circuit board in the paint, wait 20 minutes and dip again. The probe is then allowed to cure in air at room temperature for at least 48 hours. This example is for illustration as there are many possible coatings such as vacuum formed plastic shells or appropriate vapor deposited coatings Such as Parylene. In some embodiments, glass coatings are used.

In one embodiment, a separate system component is provided for human machine interface in an example of such an embodiment, a Treo or other Bluetooth enabled Palm device is used. The Palm device sends a read command to the PIC via Bluetooth and reads the array of 16 integers from the PIC. This data is shown on the Palm display as a bar graph in horizontal orientation with the top most channels at the top left of the display with increasing values expanding the bar to the right. This gives a good visual representation of the real time status of the sensor in liquid. A color threshold is implemented to show the values typically associated with air, gas, and ethanol. An air value is typically between 4.5 and 5.5 pF, gasoline is between 6.0 and 10.0, ethanol and ethanol with water and water usually maximize the output, Which is defined as 30 pF. Colors are assigned to these ranges as blue for air, green for gasoline, and red for ethanol or water.

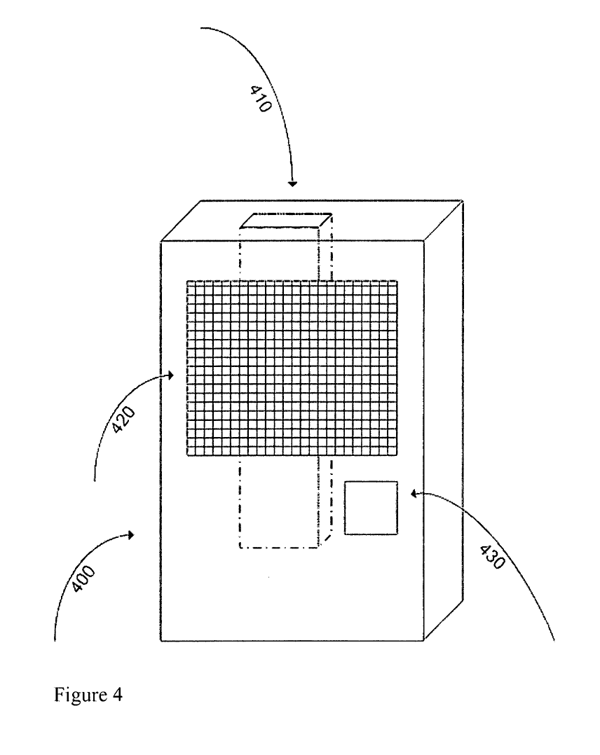

In accordance with an embodiment of the present invention as shown in FIG. 4, a user determines the volume fractions of ethanol and gasoline in a mixture by capturing in a chamber (410) within a device (400) a known volume and inducing a two-phase event in that volume by adding water into the chamber (410) and using a sensor as described herein, to determine the volume of each of ethanol with water and gasoline. Considering that one knows first the volume of ethanol/gasoline mixture and the amount of water added one can then determine by volumetric analysis the % v/v of ethanol to gasoline. Such a device (400) can be made portable for a rapid field analysis of ethanol/gasoline blends by % v/v. in an embodiment the device (400) includes electronic circuits and sensors as disclosed herein for making the measurements in accordance with the present invention. The device (400) may additionally include standard interface elements such as but not limited to a display (420) for user interaction, and a power switch (430) for device activation and shut-down.

While many of the embodiments above are described in terms of dielectric or impedance spectroscopy from capacitive probes, it will be appreciated by those of skill in the art that other systems are possible. Embodiments that use optical measurements and optical spectroscopy can accomplish similar goals, and systems designers can use the inventions disclosed herein either way, depending on the advantages brought by the corresponding techniques. An advantage of the capacitive measurements (but not the only one), is robustness in the presence of optically opaque contaminants. Additionally there are other equivalent or related techniques that can be substituted for elements of the embodiments disclosed herein, including but not limited to the use of capacitive bridge circuits comprised in part of the sensors described herein, as well as electromagnetic resonant cavity sensors that likewise allow for the measurement of dielectric properties of materials inside, to name two.

Particular uses of the various embodiments disclosed herein include, but are not limited to: measuring fuel in the fuel line of a car to generate an alarm in the case that fuel composition is such that two phasing or other water intrusion complications poses a danger; and measuring the heights of layers of one or more of air, gasoline mixtures, and water mixtures within a gasoline storage tank including but not limited to an underground storage tank at a gas station, to detect the amount of water at the bottom of the tank as well as the presence of two phasing in the tank. Additionally, Such embodiments can include more sophisticated spectral measurements that allow for the detection or absence of a predetermined concentration of ethanol in gasoline layers—to assist in the detection of two phasing events and generally to validate compliance with environmental regulations for oxygenated fuels.

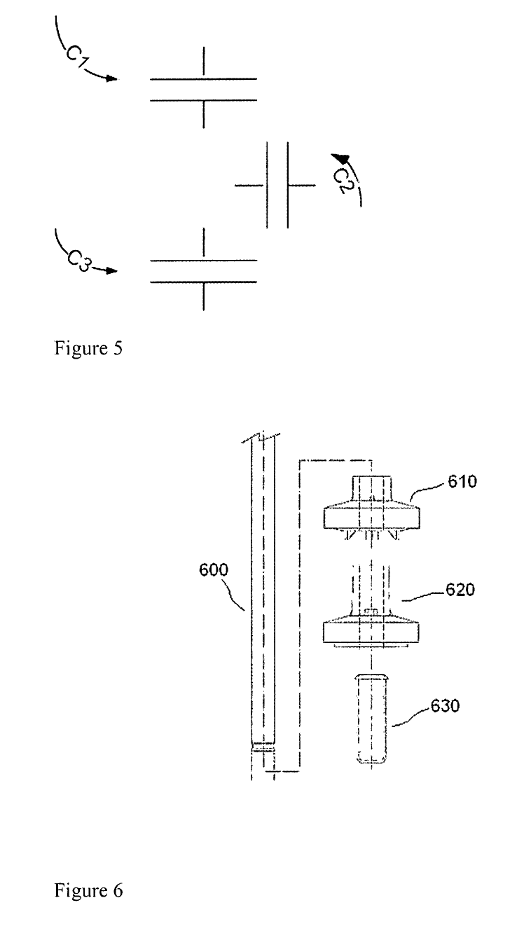

As shown in FIG. 5, in some embodiments, combining vertical and horizontal capacitors allows for more precise measurement of the relative height of the interface between 2 layers. In the figure, capacitor C1 is in layer 1, C2 is in a mixed layer, and C3 is in layer 2. The problem is that temperature, unknown additives, etc, make it such that one can not know too precisely the impedance spectrum of the individual (pure) layers a priori, and so one would have difficulty precisely numerically un-mixing the data from C2. In the embodiment shown in accordance with an aspect of the present invention, C1 and C3 measure the spectra of the pure layers, thus enabling this un-mixing, for example by linear algebraic techniques standard in the art. It is sometimes helpful in this instance to practice the form of the invention wherein additional variable series capacitors are added to the probes, in order to calibrate so that each sensor's capacitance is within the range of the capacitance measurement circuit as disclosed elsewhere herein.

In one embodiment of the present invention, a float system is used in which one or more members comprised of a float and a vertical series of capacitive sensors as disclosed herein attached to said float, is designed and disposed to float at or near the interface between two layers in a multi-layer fuel tank composition (e.g., the interface between gasoline and air at the top, or the interface between gasoline and water or water+ethanol). As describe previously herein, the presence of variable concentration of ethanol complicates the functioning of these floats, rendering straight readings from such floats problematic. The readings of the individual sensors allow for more precise knowledge of the height of the sensor compared with the interface. Current state of the art float based systems use magnetorestrictive techniques to very accurately measure the height position of the float. Given a definable range of mixture and environmental conditions, those of skill in the art will readily see how to design a float that will predictably rest within a certain distance (herein the tolerance distance) from the interface. By disposing the vertical series of capacitive sensors to extend above and below the center of the float by at least the tolerance distance, the resulting system accurately measures the position of the interface as disclosed herein. The absolute position of the center of the float will be accurately measured by conventional magnetorestrictive or other measurement, and then the distance of the fluid interface to the center of the float will be measured by the vertical series of capacitive sensors. The accuracy of the measurement will be determined by the resolution of the float position measurement combined with the resolution of the capacitive sensor array as previously discussed. These level and capacitive measurements, together with knowledge of the buoyancy of the member(s), provide additional information about the height(s) and compositions of the layers above and below the interface.

In another embodiment, a combined system has a float for the top layer and a vertical series of capacitors near the bottom of the tank, including but not limited to the case where the vertical array extends from the bottom of the tank. In this way the float can measure the height of the total volume of fluid in the tank, and the vertical capacitor series can measure the height of any layer near the bottom of the tank that is comprised of water.

In another embodiment, a sensor can be made to detect the onset or completion of a phase separation event. A single capacitive probe, which in one embodiment can be vertical and/or horizontal plates, can be placed in a tank or line pumping out of a tank such that it is submerged in the fuel but in a location known to be above any possible separation layer (i.e., always completely in gasoline layer). In the case of gasoline with ethanol additive, this would mean that the probe sits in the gasoline layer and above any water layer as well as above the level that the water-ethanol layer would reach should the fuel 2-phase. In the situation where the additive has very different dielectric properties from gasoline, as is the case with Ethanol, this measurement easily detects and correlates to the additive's concentration in the gasoline. With appropriate environmental measurements (pressure, temperature, etc.) and a precomputed model, the precise concentration of the additive can be calculated. One can monitor this value over time and when a substantially sudden drop below an acceptable threshold value is detected, the phase separation of the additive is known to be happening or to have already happened. In another embodiment, starting with initial measurements made when the fuel is assumed to have not separated and to still contain the full concentration of additive such as Ethanol, the measured dielectric of the gasoline layer is tracked. Without calculating the precise concentration of the additive, one simply monitors the measured value and when it drops below a well-tested threshold relative value (i.e., a percentage of a recent measured average value taken when fuel was assumed to not have separated), the 2-phasing event can be assumed to be occurring or to already have occurred.

In another embodiment, the system can be used to determine 1) the height of any separation layer at the bottom of a tank (i.e., water or water and ethanol layer) and 2) whether a 2-phasing of the fuel in the tank has occurred. A vertical array of capacitive probes, which can be but are not limited to vertical and/or horizontal plates, are arranged in a vertical series as in previously discussed configurations. Because the dielectric properties of gasoline vs. water and or Ethanol are a different order of magnitude, the probes and measuring circuit can be designed not to have to precisely quantitatively measure but rather to function as a binary detector that only distinguishes between gasoline and an unspecified mixture of water and ethanol, essentially a switch that detects the presence of a high dielectric. A precise capacitor in series for each probe will limit the value detected in water or ethanol to a known value; the measured gasoline value will be significantly less and a threshold value can be chosen to be reliable under all realistic environmental conditions. To measure the height the layer at the bottom, this vertical array must contain enough of these “switches” sampling at enough locations to achieve the desired spatial resolution. For very high resolution, where a large number of densely packed probes would be required, staggered or helical geometries can be used to deploy the probes. Furthermore, the measurement circuits could apply some coding schemes, analog switching, or perhaps subsets of the probes can be hooked up as capacitors in parallel, so that fewer measurements need be made and due to the discrete nature of the “switch” the number of “ons” can be determined. This system can be designed to work very robustly under realistic environmental conditions. More generally, the system can be applied in any situation where a phase layer in a fluid or gas mixture needs to be detected or measured, when the constituents of each layer have very different dielectric properties.

It should be appreciated that the forgoing discloses some embodiments for systems that detect fuel quality problems in fuel storage tanks, such systems comprising the steps of providing a plurality of sensors at different heights within the tank, measuring a property of the fuel with some of the sensors to obtain height dependent measurements, using the height dependent measurements to determine a property of the contents of the tank, wherein the property is thereby determined at a plurality of heights within the tank and the property and measurements provide some information about fuel quality and problems with fuel quality.

It should be noted that certain of the embodiments of the kind described in the previous paragraph relate to the measurement of buoyancy of fluid at certain levels within a tank. In particular, one embodiment consists of a float that is such that it will float on water but will not float in gasoline (which is less dense than water). Such a float can be used, for example near the bottom of a gas tank, to determine when water has leaked into the gasoline, or separated from the gasoline for some reason. However, impurities in fuel can adhere to such a float, and can also create a layer of impurity at the bottom of the tank that will confuse such a sensor. Also, flexible fuels and other blended fuels may have additives that affect the density of the fuel or the various layers. Consequently, there is a need for embodiments that can measure properties of fuel, and sometimes properties of layers of liquids within the fuel, beyond the physical property of buoyancy. In accordance with an aspect of the present invention, the devices, methods and systems disclosed herein are used to acquire chemical composition information about the fuel at various locations within a fuel system (these locations including but not limited to different heights). This information is more informative and useful than simple buoyancy measurements, and allows addressing of the problem just described. In particular, the information provides information about the composition of fluids and/or layers within the fuel. It can enable chemical analysis of the fuel and thereby provide for assessment and monitoring fuel integrity.

The methods, systems and devices disclosed herein can be used to assess and monitor fuel quality at all points along the chain of production, distribution and use of fuel—from the refinery to tankers and pipelines to intermediate storage facilities to delivery trucks to gas station storage tanks to automobile fuel tanks and other parts of the fuel system within automobiles. Moreover, they can be used to assess and monitor other fuels including but not limited to commercial and residential heating oil (for example stored in basement, underground, or outside heating oil storage tanks).

An embodiment is comprised of a pair of ceramic circuit boards, with a predetermined number (say 256) of individual electrodes (corresponding pairs facing each other on the two boards). Each pair would then comprise an individually addressable impedance sensor. In one such embodiment, a large number of wires would be need to connect these 512 electrodes to external electronics, in another embodiment, some of the electronics is placed on or near the ceramic circuit boards (can be part of the ceramic boards), so that multiplexing can be done and a smaller cable exiting (addressing and impedance measurement circuits can be local, and say serial data sent over a smaller cable, transmitting to a controller unit that can be outside the tank). In some embodiments, the electronics components other than the electrodes, that are inside the tank, are potted (an electronics encapsulation technique that is a term of art in electrical engineering wherein parts are enclosed in resins or other materials so as to be mechanically and physically isolated). Some embodiments additionally are comprised of a temperature sensor inside the tank, or at or near some of the electrodes. Temp and current measurements for safety—power limiting as a safety feature.

Another embodiment of the present invention is comprised of a prior art flexible fuel sensor such as a Ford flexible fuel sensor part number YL5A-9C044-AA, and further comprised of a component for passing electrical signals through the flex fuel sensor and processing the response signals in accordance with the techniques disclosed herein, in order to measure a property of the fuel passing through the sensor, including but not limited to the presence or amount of water or other contaminant(s) present in the fuel.

Another embodiment of the present invention is comprised of a capacitive and/or impedance and/or optical spectral measurement component disposed to measure fuel at a fuel dispenser Such as a gas station pump, and further comprised of a component to process signals from the sensor(s) in accordance with the techniques disclosed herein, in order to measure a property of the fuel passing through the sensor, including but not limited to the presence or amount of water or other contaminants(s) present in the fuel.

FIG. 6 shows prior art components for fuel monitoring in which a probe shaft (600) is deployed substantially vertically within a fuel tank. A “product float” (610) slides on the shaft (600) and the height of the float (610) is read, for example by a magnetorestrictive sensor, to give the level of total product (such as gasoline) plus water in the tank. A second optional water float (620) is similarly disposed on the shaft, and floats on a water layer within the tank, similarly yielding an estimate of the water layer within the tank. Finally, a boot (630) to seal off or protect the probe shaft is placed at the end of the shaft (600).

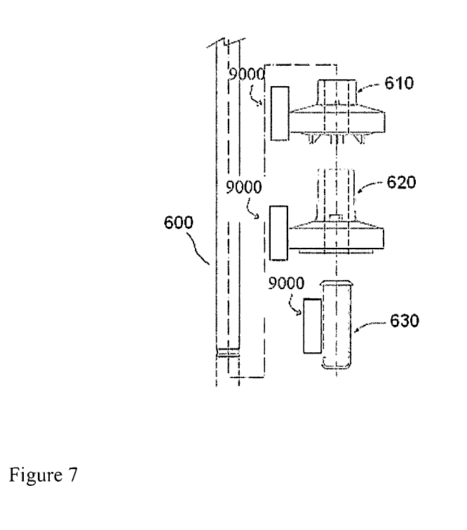

FIG. 7 shows an embodiment in accordance with the present invention, comprised of modification of the components from FIG. 6. FIG. 7 shows various locations, labeled collectively by (9000), at which a sensor in accordance with the present invention, can be attached to the components shown in FIG. 6. This modified component can then be used as disclosed herein to practice the invention. The locations shown are meant to be illustrative and not limiting, and one of skill in the art will readily see that other placements are possible. In some embodiments the component (9000) is comprised of a vertical series of pairs of plates, such that the pairs of plates are used as capacitive or impedance sensors in accordance with the techniques disclosed herein. Some embodiments are similarly comprised of optical sensors. The locations of the components (9000) in FIG. 7 are meant to be illustrative and not limiting. For example, such a component (9000) could be located below the “boot” component in the figure, even though this location is not shown in FIG. 7.

An embodiment of the present invention comprises a gasoline monitoring device and system that is part of a fuel dispenser. In such an embodiment, fuel is monitored, for example, within a gas station gasoline dispenser in accordance with the methods, devices and systems disclosed herein. The invention is particularly advantageous in this regard because the flowing, often turbulent gasoline that passes through a dispenser is not disposed to be easily measured by purely mechanical systems, methods and devices, but is much more amenable to measurement by spectral systems, methods and devices including but not limited to optical, electrical, impedance and/or capacitance spectral measurements is accordance with the present invention.

The devices, methods and systems disclosed herein relate to electromagnetic measurements comprised of optical, capacitance and impedance measurements. It should be understood that he methods disclosed can use various combinations of these electromagnetic measurements.

While the foregoing has described and illustrated aspects of various embodiments of the present invention, those skilled in the art will recognize that alternative components and techniques, and/or combinations and permutations of the described components and techniques, can be substituted for, or added to, the embodiments described herein. It is intended, therefore, that the present invention not be defined by the specific embodiments described herein, but rather by the claims, which are intended to be construed in accordance with the well-settled principles of claim construction, including that: each claim should be given its broadest reasonable interpretation consistent with the specification; limitations should not be read from the specification or drawings into the claims; words in a claim should be given their plain, ordinary, and generic meaning, unless it is readily apparent from the specification that an unusual meaning was intended; an absence of the specific words “means for” connotes applicants' intent not to invoke 35 U.S.C. §112 (6) in construing the limitation; where the phrase “means for” precedes a data processing or manipulation “function,” it is intended that the resulting means-plus-function element be construed to cover any, and all, computer implementation(s) of the recited “function”; a claim that contains more than one computer-implemented means-plus-function element should not be construed to require that each means-plus-function element must be a structurally distinct entity (such as a particular piece of hardware or block of code); rather, such claim should be construed merely to require that the overall combination of hardware/firmware/software which implements the invention must, as a whole, implement at least the function(s) called for by the claim's means-plus-function element(s).

1. A method for determining fuel quality in a fuel storage tank comprising the steps of:
providing a plurality of sensors at different heights within said fuel storage tank;
measuring a property of the fuel in said fuel storage tank with at least some of said sensors to obtain height dependent measurements; and
determining said fuel quality of the fuel in said fuel storage tank at a plurality of heights within said fuel storage tank using said height dependent measurements.

2. The method of claim 1, wherein the step of determining said fuel quality determines said fuel quality of the fuel in said fuel storage tank at predetermined heights within said fuel storage tank.

3. The method of claim 1, wherein the step of determining said fuel quality determines said fuel quality of the fuel in said fuel storage tank at heights within said fuel storage tank relative to a system component.

4. The method of claim 3, wherein the step of determining said fuel quality determines said fuel quality of the fuel in said fuel storage tank at heights within said fuel storage tank relative to a buoyant element.

5. The method of claim 1, wherein the step of providing a plurality of sensors provides a plurality of impedance sensors at different heights within said fuel storage tank.

6. The method of claim 1, wherein the step of providing a plurality of sensors provides a plurality of spectral sensors at different heights within said fuel storage tank.

7. The method of claims 1, wherein the step of measuring a property measures said property of the fuel in a gas station fuel storage tank.

8. The method of claim 7, wherein the step of determining said fuel quality comprises the step of detecting presence or absence of a two phasing event.

9. The method of claim 7, wherein the step of determining said fuel quality comprises the step of determining a likelihood of a two phasing event.

10. The method of claim 5, further comprising the step of driving said plurality of impedance sensors with a plurality of AC frequencies.

11. The method of claim 10, wherein the step of measuring said property comprises the step of measuring impedance spectroscopy of the fuel in said fuel storage tank.

12. The method of claim 11, further comprising the step of estimating material composition of the fuel in said fuel storage tank based on said impedance spectroscopy measurements.

13. The method of claim 11, wherein the step of measuring said property comprises the step of measuring optical spectroscopy of the fuel in said fuel storage tank.

14. The method of claim 6, wherein the step of measuring said property comprises the step of measuring optical spectroscopy of the fuel in said fuel storage tank.

15. The method of claim 1, wherein the step of measuring said property measures the property of the fuel in a portion of said fuel storage tank; and wherein the step of determining said fuel quality determines said fuel quality of the fuel in said portion of said fuel storage tank.

16. The method of claim 1, wherein the step of measuring said property measures the property of the fuel in a bottom portion of said fuel storage tank; and wherein the step of determining said fuel quality determines said fuel quality of the fuel in said bottom portion of said fuel storage tank.
